SEQUENCE LISTING (7 sequences)

SEQ ID NO: 1 - DNA, 36 bp, synthetic construct
  source 1..36: mol_type = other DNA; note = MN4
ggcgacaaggaaaatccttcaacgaagtgggtcgcc

SEQ ID NO: 2 - DNA, 24 bp, synthetic construct
  source 1..24: mol_type = other DNA; note = the third base sequence (MN4)
aaggaaaatccttcaacgaagtgg

SEQ ID NO: 3 - DNA, 30 bp, synthetic construct
  source 1..30: mol_type = other DNA; note = MN6
gacaaggaaaatccttcaatgaagtgggtc

SEQ ID NO: 4 - DNA, 30 bp, synthetic construct
  source 1..30: mol_type = other DNA; note = MN19
gacaaggaaaatccttcaacgaagtgggtc

SEQ ID NO: 5 - protein, 4 aa, synthetic construct
  source 1..4: note = polycation
KKKK

SEQ ID NO: 6 - protein, 5 aa, synthetic construct
  source 1..5: note = polycation
KKKKK

SEQ ID NO: 7 - DNA, 11 bp, synthetic construct
  source 1..11: mol_type = other DNA; note = the second nucleotides
gagagatggcg

(19) United States
(12) Patent Application Publication
(10) Pub. No.: US 2024/0110229 A1
(43) Pub. Date: Apr. 4, 2024

(54) CHEMICAL SENSOR USING STRAND EXCHANGE REACTION

(71) Applicant: KABUSHIKI KAISHA TOSHIBA, Tokyo (JP)

(72) Inventors: Yoshiaki SUGIZAKI, Fujisawa (JP); Hiroko MIKI, Kawasaki (JP)

(73) Assignee: KABUSHIKI KAISHA TOSHIBA, Tokyo (JP)

(21) Appl. No.: 18/184,086

(22) Filed: Mar. 15, 2023

(30) Foreign Application Priority Data
Sep. 16, 2022 (JP) 2022-148522

(51) Int. Cl. C12Q 1/6825; C12Q 1/6876

(52) U.S. Cl. CPC C12Q 1/6825; C12Q 1/6876; C12Q 2600/136

(57) ABSTRACT

According to one embodiment, a chemical sensor including a nucleic acid probe for capturing a target substance, a sensor element that has a surface on which the nucleic acid probe is immobilized, and a liquid film that covers the sensor element is provided.

### CROSS-REFERENCE TO RELATED APPLICATIONS

This application is based upon and claims the benefit of priority from Japanese Patent Application No. 2022-148522, filed Sep. 16, 2022, the entire contents of which are incorporated herein by reference.

### REFERENCE TO A SEQUENCE LISTING

In accordance with 37 CFR § 1.831, the present specification makes reference to a Sequence Listing submitted electronically as a .xml file named “Sequence Listing.xml”. The .xml file was generated on Mar. 15, 2023 and is 8,272 bytes in size. The entire contents of the Sequence Listing are hereby incorporated by reference.

### FIELD

Embodiments described herein relate generally to a chemical sensor using a strand exchange reaction.

### BACKGROUND

There is a need for a chemical sensor including a nucleic acid probe that can be measured with high sensitivity.

### BRIEF DESCRIPTION OF THE DRAWINGS

FIG. 1 is a cross-sectional view illustrating an example of a chemical sensor of a first embodiment.

Part (a) of FIG. 2 is a schematic view illustrating an example of a nucleic acid probe of the chemical sensor of the first embodiment, part (b) of FIG. 2 is a view illustrating progress of a strand exchange reaction in the nucleic acid probe, and part (c) of FIG. 2 is a view illustrating a nucleic acid probe in which the strand exchange reaction is completed and a stable higher-order structure is formed.

FIG. 3 is an energy level diagram relating to the nucleic acid probe included in the chemical sensor of the first embodiment, and illustrates a change in free energy between a case where the nucleic acid probe forms a double strand and a case where the nucleic acid probe forms a single strand.

Part (a) of FIG. 4 is a view illustrating an example of a second nucleic acid containing a polycation included in a chemical sensor of a second embodiment, and part (b) of FIG. 4 is a schematic view illustrating a state in which the second nucleic acid containing a polycation and another base sequence are assembled in a strand exchange reaction.

FIG. 5 is an energy level diagram relating to a nucleic acid probe included in the chemical sensor of the second embodiment, and illustrates a change in free energy due to capture of a target substance.

FIG. 6 is a cross-sectional view illustrating an example of a chemical sensor of a third embodiment.

Part (a) of FIG. 7 is a view illustrating an example of a polycation included in the chemical sensor of the second embodiment, and part (b) of FIG. 7 is a schematic view illustrating a state in which the polycation and another base sequence are assembled in a strand exchange reaction.

FIG. 8 is a flowchart of an analysis method using a chemical sensor according to a fourth embodiment.

FIG. 9 is a flowchart of a method for manufacturing a chemical sensor according to a fifth embodiment.

FIG. 10 is a graph showing a temporal change in drain current flowing through a sensor element when a concentration of a target substance is changed stepwise among the measurement results of Example 1.

FIG. 11 illustrates gate voltage dependency (“IdVg characteristic”) of the drain current of an FET sensor including a graphene film during each measurement period among the measurement results of Example 1.

### DETAILED DESCRIPTION

In general, according to one embodiment, a nucleic acid probe for capturing a target substance, a sensor element that has a surface on which the nucleic acid probe is immobilized, and a liquid film that covers the sensor element are provided. The nucleic acid probe is a double-stranded nucleic acid composed of a first nucleic acid and a second nucleic acid bound to the first nucleic acid, and the first nucleic acid is a nucleic acid containing a first base sequence, a second base sequence consisting of a base sequence complementary to the first base sequence, and a third base sequence having one end bound to the first base sequence and the other end bound to the second base sequence. The second nucleic acid is a nucleic acid containing a fourth base sequence consisting of a base sequence complementary to a part of the second base sequence of the first nucleic acid, in which the fourth base sequence binds to the second base sequence of the first nucleic acid. The third base sequence includes a base sequence constituting a binding site that captures a target substance.

Hereinafter, embodiments will be described with reference to the accompanying drawings. Note that, in each of the embodiments, substantially the same constituents are denoted by the same reference numerals, and the description thereof may be partially omitted. The drawings are schematic, and a relationship between a thickness and a planar dimension of each part may be actually different from a ratio of the thickness of each part.

### First Embodiment

(Chemical Sensor)

According to a first embodiment, there is provided a chemical sensor including a nucleic acid probe that detects a target substance by a strand exchange reaction (hereinafter, referred to as a “sensor 1”). The sensor 1 includes a sensor element 2 and a liquid film 3 disposed to cover the sensor element 2, and a nucleic acid probe 4 is immobilized on a surface of the sensor element 2.

Any type of sensor element may be selected as the sensor element 2 as long as it is configured to be sensitive to a change in secondary structure of the nucleic acid probe 4 described below. The sensor element 2 may be, for example, a graphene field-effect transistor (GFET), an ion sensitive field-effect transistor (ISFET), a surface plasmon resonance device (SPR), or a quartz crystal microbalance (QCM).

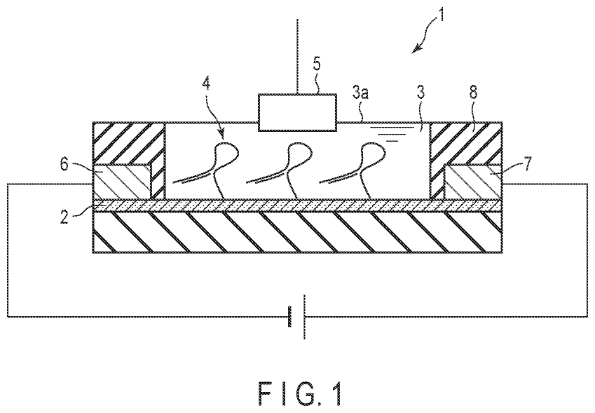

For example, in a case where the sensor 1 is a type of FET sensor, as illustrated in FIG. 1, the sensor element 2 is disposed so as to be in contact with a gate electrode 5 through the liquid film 3, and a source electrode 6 and a drain electrode 7 are electrically connected to one end and the other end of the sensor 1, respectively. In addition, a circuit that applies a voltage (that is, a gate voltage) is connected to the gate electrode 5. A circuit that applies a voltage is also formed between the source electrode 6 and the drain electrode 7, and an ammeter (not illustrated) that measures a drain current flowing on the circuit is disposed. The source electrode 6 and the drain electrode 7 may be covered with an insulating protective film 8.

The liquid film 3 is disposed so that a surface 3a thereof is in contact with a specimen sample containing a target substance, covers the sensor element 2, and is disposed so that the nucleic acid probe 4 immobilized on a surface 2a of the sensor element 2 is immersed. The liquid film 3 is formed of a measurement solution capable of dissolving the target substance. For example, water can be selected as a solvent of the measurement solution, and any reagent (for example, a stabilizer, a pH adjusting agent, ions, or the like) required for measurement or storage of the sensor 1 may be contained as a solute.

The nucleic acid probe 4 is a double-stranded nucleic acid composed of a first nucleic acid and a second nucleic acid complementary to the first nucleic acid, and has a binding site for capturing a target substance. Here, the first nucleic acid is a nucleic acid containing a first base sequence; a second base sequence consisting of a base sequence complementary to the first base sequence; and a third base sequence having one end bound to the first base sequence and the other end bound to the second base sequence. In addition, the second nucleic acid is a nucleic acid containing a fourth base sequence consisting of a base sequence complementary to a part of the second base sequence of the first nucleic acid, in which a part of the fourth base sequence binds to the second base sequence of the first nucleic acid. Here, the third base sequence includes a base sequence constituting a binding site that captures a target substance.

One of the first nucleic acid and the second nucleic acid is immobilized on the surface of the sensor element, such that the nucleic acid probe 4 is immobilized on the surface 2a of the sensor element 2. The immobilization of the nucleic acid probe 4 can be performed by making a material constituting the surface 2a of the sensor element 2 to be a material capable of binding to the first nucleic acid or the second nucleic acid of the nucleic acid probe 4. For example, when the sensor element 2 is formed of graphene or gold, the nucleic acid probe 4 is immobilized because of non-specific adsorption with the nucleic acid. More preferably, a functional group that binds to or absorbs a material constituting the surface 2a of the sensor element 2 is modified on the nucleic acid probe 4, and the nucleic acid probe 4 may be immobilized by binding to or adsorbing the sensor element surface 2a and the functional group. For example, in a case where the sensor element 2 is graphene, when a polycyclic aromatic system, for example, pyrene is modified as the functional group, the nucleic acid probe 4 is strongly immobilized on the graphene sensor element surface 2a by a π-π interaction between the polycyclic aromatic system and the graphene. Alternatively, in a case where the sensor element 2 is gold, when a thiol group is modified as the functional group, the thiol group strongly binds to gold, such that the nucleic acid probe 4 is strongly immobilized on the gold surface 2a of the sensor element 2. Note that the functional group modification of the nucleic acid may be performed by nucleic acid synthesis by a phosphoramidite method using an amidite reagent having a desired functional group. Since the nucleic acid probe 4 is immobilized on the surface 2a of the sensor element 2, a structural change or release of the nucleic acid probe 4 is detected as a signal change of the sensor element 2. For example, in a case where the sensor element is a GFET or an ISFET, since the proximity or separation of the nucleic acid probe 4 having a negative charge in a phosphate ester main skeleton portion is applied as a gate voltage to the FET, a change in the drain current is detected. Alternatively, in a case where the sensor element is an SPR, the SPR detects a change in dielectric constant on the sensor element surface due to dissociation of the first nucleic acid and the second nucleic acid of the nucleic acid probe 4 and release of the nucleic acid that is not immobilized on the sensor element surface as a change in resonance effect with surface plasmon. Alternatively, in a case where the sensor element is a QCM, the QCM detects a change in the amount of nucleic acid immobilized on the sensor element surface due to dissociation of the first nucleic acid and the second nucleic acid of the nucleic acid probe 4 and release of the nucleic acid that is not immobilized on the sensor element surface as a change in resonance frequency of a crystal resonator.

The nucleic acid probe 4 is a double-stranded nucleic acid composed of the first nucleic acid and the second nucleic acid as described above, and can be denatured so that the first nucleic acid and the second nucleic acid are dissociated by a strand exchange reaction described below.

After the first nucleic acid constituting the nucleic acid probe 4 is dissociated from the second nucleic acid, a secondary structure thereof is autonomously changed so as to increase thermodynamic stability. Since the first nucleic acid contains the first base sequence and the second base sequence complementary to each other in a molecule thereof, the first nucleic acid autonomously forms a secondary structure having a stem portion formed by pairing the first base sequence and the second base sequence and a loop portion or a bulge structure containing the third base sequence after dissociating from the second nucleic acid. Examples of the secondary structure autonomously, which is formed by the second nucleic acid, of the dissociated first nucleic acid include a hairpin loop, an internal loop, a bulge, a pseudoknot, a 3 way junction (3WJ), and a 4 way junction (4WJ).

It is preferable that the first nucleic acid having a secondary structure is more stable. That is, a melting temperature Tm of the first nucleic acid having the stem portion formed by pairing the first base sequence and the second base sequence is preferably higher. The melting temperature Tm can be calculated by any calculation method such as a basic calculation method or the most proximal base pair method. A value of a melting temperature Tm1 of the double-stranded nucleic acid formed by pairing the nucleic acid containing the first base sequence and the nucleic acid containing the second base sequence is higher than a value of a melting temperature Tm2 of the double-stranded nucleic acid formed by pairing the nucleic acid containing the first base sequence and the nucleic acid containing the fourth base sequence. Tm1 and Tm2 do not consider melting of the secondary structure formed by the third base sequence. The first base sequence and the second base sequence are complementary between, for example, 4 to 12 bases. The first base sequence and the second base sequence have, for example, 4 to 12 bases.

The second nucleic acid constituting the nucleic acid probe 4 is a nucleic acid containing the fourth base sequence as a full length or a part thereof. In a case where the second nucleic acid contains the fourth base sequence as a partial sequence thereof, the fourth base sequence may not be completely complementary to the second base sequence of the first nucleic acid. For example, the fourth base sequence may include a base sequence mismatched with the second base sequence, or may include a base sequence part that does not pair with the second base sequence and forms a bulge structure. Conversely, the second base sequence of the first nucleic acid may include a base sequence part that does not pair with the fourth base sequence and forms a bulge structure. For example, the fourth base sequence has a base length shorter than that of the second base sequence.

The denaturation of the nucleic acid probe 4 can be caused by a strand exchange reaction that dissociates the first nucleic acid and the second nucleic acid. As described below, the strand exchange reaction in the nucleic acid probe 4 of the present embodiment is a reaction in which the binding of the fourth base sequence of the second nucleic acid and the second base sequence of the first nucleic acid is gradually replaced by the binding of the first base sequence of the first nucleic acid and the second base sequence of the first nucleic acid, and such a strand exchange reaction can be induced and enhanced by capturing a target substance at a binding site constituted by the third base sequence of the first nucleic acid of the nucleic acid probe 4.

In addition, as described below, the base length of the base sequence present between the first base sequence (or the second base sequence) and the base sequence constituting the binding site in the third base sequence is preferably shorter. However, a spacer composed of one base or a spacer sequence composed of two bases or three bases may be contained between the second base sequence and the base sequence constituting the binding site of the third base sequence. In addition, a spacer composed of one base may be contained between the first base sequence and the base sequence constituting the binding site of the third base sequence.

The first nucleic acid dissociated from the second nucleic acid and having a secondary structure that is autonomously stably formed may be, for example, MN4 (SEQ ID NO: 1: GGCGACAAGGAAAATCCTTCAACGAAGTGGGTCGCC). MN4 is an anti-cocaine aptamer capable of capturing cocaine as a target substance, but can also capture methyl benzoate, which is an impurity in a cocaine production process and is also an odor component of cocaine, and has a secondary structure as shown in the following Formula 1-02. A dissociation constant for cocaine of MN4 is 7±1 μM and a dissociation constant for methyl benzoate is 87.7±12.5 μM.

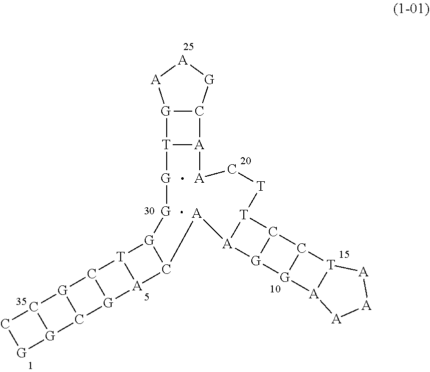

In a case where the first nucleic acid is MN4, the first base sequence constituting the first nucleic acid is a sequence arranged in the order of GGCGAC from the 5′ end of MN4 in the 5′→3′ direction, the second base sequence is a sequence arranged in the order of CCGCTG from the 3′ end of MN4 in the 3′→5′ direction, and the third base sequence is a sequence arranged in the order of AAGGAAAATCCTTCAACGAAGTGG (SEQ ID NO: 2) in the 5′→3′ direction.

In the above case, the fourth base sequence and the second nucleic acid may be, for example, a sequence arranged in the order of GCGA, a sequence arranged in the order of GCGAC, or a sequence arranged in the order of GGCG, from the 5′ end to the 5′→3′ direction.

On the other hand, as other nucleic acid constructs having a function of an anti-cocaine aptamer, MN6 (SEQ ID NO: 3: GACAAGGAAAATCCTTCAATGAAGTGGGTC) and MN 19 (SEQ ID NO: 4: GACAAGGAAAATCCTTCAACGAAGTGGGTC) are known. MN6 and MN19 have secondary structures represented by the following Formulas 1-02 and 1-03, respectively.

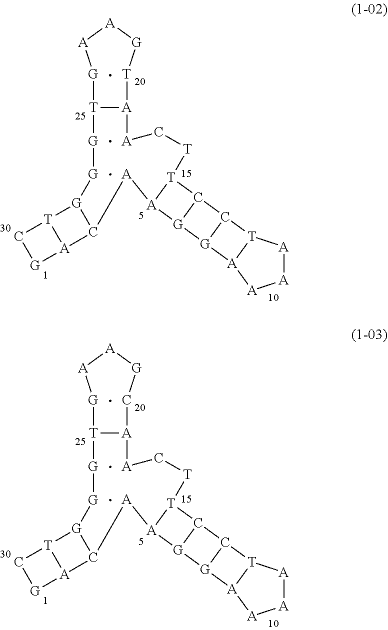

MN6 and MN19 are unstable nucleic acid constructs showing an equilibrium state between a state in which base sequences corresponding to the first base sequence and the second base sequence are autonomously combined to form a secondary structure and a state in which the base sequences corresponding to the first base sequence and the second base sequence are not combined to have no secondary structure. Furthermore, a dissociation constant between MN6 and cocaine is 45.3±0.5 μM, the dissociation constant for MN19 is 26.7±0.7 μM, and thus the ability to bind to a target substance and to capture the target substance is low. Therefore, MN6 and MN19 are unstable and difficult to handle when used as aptamers as compared with MN4. Furthermore, the dissociation constant of them with methyl benzoate as an odor component of cocaine cannot be detected by isothermal titration calorimetry (ITC). Therefore, MN6 and MN19 cannot be used as aptamers for methyl benzoate in ITC.

However, MN6 and MN19 have a feature that a change in secondary structure occurs, which is triggered by the capture of cocaine as a ligand. Since a main skeleton of the nucleic acid has a negative charge, such a change in secondary structure can detect a relatively large signal from the sensor element as proximity or separation of the negative charge. Therefore, MN6 and MN19 have excellent functions as a nucleic acid probe of a chemical sensor.

As described above, although MN4 has high affinity with a target substance as an anti-cocaine aptamer, a stable secondary structure is formed without capturing the target substance. A problem is that a change in higher-order structure, that is, a change in sensor signal caused by capturing a target substance by MN4 is small.

Therefore, it has been a problem that it is difficult to achieve both a high binding ability and a stable structure (desirable as an aptamer of a target substance) and a high signal output due to a large change in the higher-order structure (desirable as a nucleic acid probe). Furthermore, at a concentration lower than the dissociation constant, the binding frequency is decreased, and thus there is also a problem in principle that detection is difficult.

However, the inventors have recently found that the same problem can be solved by using a nucleic acid having a metastable structure as a probe for capturing a target substance, metastable structure formed by binding a short-chain complementary chain as described below. Hereinafter, the metastable structure of the nucleic acid probe 4 will be described with reference to FIGS. 2 to 4.

Part (a) of FIG. 2 illustrates a secondary structure before the strand exchange reaction is caused in the nucleic acid probe 4 of the present embodiment. That is, the secondary structure of the double-stranded nucleic acid formed by binding of the short-chain complementary chain illustrated in part (a) of FIG. 2 is a metastable structure. Note that although MN4 will be described as an example of the nucleic acid probe 4 (see FIG. 2) of the present embodiment, a base sequence other than MN4 is also expected to have the same mechanism of action as described below. For example, the first base sequence and the second base sequence are at least complementary to each other, and the sequences may be different from MN4. Note that in a case where the first base sequence is changed, the fourth base sequence also needs to be a complementary sequence corresponding thereto.

When the change in free energy due to the formation of the metastable structure illustrated in part (a) of FIG. 2 is calculated as a free energy at the time of formation of a double strand of the first base sequence and the fourth base sequence, it is about −8.33 kcal/mol, and the chemical stability of the nucleic acid probe 4 having a metastable structure is relatively excellent (note that the value of the free energy having a metastable structure can vary depending on the base length, the binding position, the GC ratio, and the mismatch ratio of the complementary strand formed by the second base sequence of the first nucleic acid and the fourth base sequence of the second nucleic acid). When the target substance is captured by the nucleic acid probe 4 having a metastable structure having relatively excellent chemical stability, a strand exchange reaction described below can be caused.

The strand exchange reaction in the nucleic acid probe 4 of the present embodiment is a reaction in which the binding of the fourth base sequence of the second nucleic acid and the second base sequence of the first nucleic acid is gradually replaced by the binding of the first base sequence of the first nucleic acid and the second base sequence of the first nucleic acid. The shape of the binding site is formed by capturing a target in the base sequence part constituting the binding site in the third base sequence, and as a result, the 3′ side of the first base sequence and the 5′ side of the second base sequence are proximate to each other. Thereafter, the strand exchange proceeds to the double strand of the first base sequence and the second base sequence with this part as a starting point. Part (b) of FIG. 2 illustrates a state in which a target substance 9 is captured at the binding site and thus a strand exchange reaction proceeds.

The free energy of the nucleic acid probe 4 in a state where the strand exchange reaction proceeds has a value higher than the free energy of the metastable structure. Although it is complicated and difficult to calculate the free energy of the nucleic acid probe 4 in a state where the strand exchange reaction proceeds, it is necessary to bring the double strand of the second base sequence and the fourth base sequence having a negative charge and the first base sequence also having a negative charge proximate to each other against the Coulomb repulsive force, and thus it is apparent that the metastable state in which they are released from each other is more stable.

Although it is difficult to calculate the free energy of the nucleic acid probe 4 in a state where the strand exchange reaction proceeds, the change in free energy at the time of forming a state of starting the strand exchange reaction can be calculated as follows. In order to cause the strand exchange reaction, as illustrated in part (a) of FIG. 2, the 5′ side of the second base sequence needs to be proximate to the binding site sequence of the target. When the 5′ side of the second base sequence is proximate to the binding site sequence of the target, the 3′ side of the first base sequence is proximate when the three-dimensional structure of the binding site is formed by capturing the target, and thus the strand exchange reaction is started. Here, the state in which the 5′ side of the second base sequence is proximate to the binding site sequence of the target, which is a previous stage of forming the binding site by capturing the target, is a state in which the base sequence of the 3′ side of the target binding site sequence of the third base sequence forms a stem loop structure.

when the stem loop was formed was, the value of the free energy change was calculated as −2.92 kcal/mol, which was extremely close to 0. Here, since the calculated the base sequence site used for the calculation is different between the free energy in the strand exchange start state and the free energy in the metastable structure, it cannot be simply treated as a change in free energy, but at least a state where the strand exchange starts means that it is not as stable as forming a metastable structure.

Therefore, it is suggested that the instability of the strand exchange start state can be a barrier for stabilizing the metastable structure. Therefore, for convenience, the difference between the free energy of the metastable structure and the free energy of the strand exchange reaction start state was defined as ΔE₁.

However, as described above, ΔE₁ is not an energy barrier for causing a strand exchange reaction. Precisely, ΔE₁ corresponds to an energy barrier for completely dissociating the fourth base sequence from the double strand of the metastable structure to start the formation of the double strand with the first sequence. In the actual strand exchange, the dissociation of the initial complementary strand and the binding of the new complementary strand occur simultaneously, and thus the energy barrier becomes a little smaller. However, as described above, it is necessary to overcome a state against the Coulomb repulsive force, that is, a state where the free energy is large, and thus the nucleic acid probe 4 is inhibited from autonomously generating the strand exchange reaction.

Here, when the target molecule is captured, the 5′ side of the second base sequence and the 3′ side of the first base sequence are forcibly brought proximate to each other by forming the three-dimensional structure of the binding site. This will aid in the work of bringing the 5′ side of the second base sequence and the 3′ side of the first base sequence proximate to each other against the Coulomb repulsive force, such that the energy barrier is lower than in the case of the absence of a target. That is, the strand exchange is likely to occur due to the capture of the target substance. In addition, since the capture of the target substance only needs to be a trigger for starting the strand exchange, it is not always necessary to keep the capture state. Therefore, even when the concentration of the target substance is lower than the dissociation constant, the action as a trigger for strand exchange can be obtained.

Here, the metastable structure is formed in order to inhibit the first nucleic acid from autonomously forming a stem between the first and second base sequences. As described above, since the binding of the first base sequence and the second base sequence is started by the proximity between the 3′ side of the first base sequence and the 5′ side of the second base sequence, when the 5′ side of the second base sequence to which the fourth base sequence is bound as a double strand is shifted to the 3′ side more greatly than the 5′ side of the second base sequence to which the first base sequence is bound as a double strand, the inhibitory effect on the formation of the first and second stems of the metastable structure cannot be obtained.

However, as described below, in Examples, the present inventors have confirmed that the base on the 5′ side of the second base sequence to which the fourth base sequence is bound may be shifted to the 3′ side by one base, two bases, or three bases from the base on the 5′ side of the second base sequence to which the first base sequence is bound. In addition, since the strand exchange reaction enhanced by the target substance is started by bringing the 3′ side of the first base sequence and the 5′ side of the second base sequence proximate to each other along with the formation of the binding site, at least one of the 3′ side of the first base sequence and the 5′ side of the second base sequence is preferably close to the binding site. In this regard, the present inventors have found that a spacer composed of one base may be contained between the first base sequence and the base sequence constituting the binding site in the third base sequence.

As illustrated in part (c) of FIG. 2, the nucleic acid probe 4 after the strand exchange reaction has completely proceeded has a stem portion in which the first base sequence and the second base sequence in the first nucleic acid are paired, and exhibits a chemically significantly stable structure. The change in free energy when the first base sequence and the second base sequence form a stem is −12.74 kcal/mol.

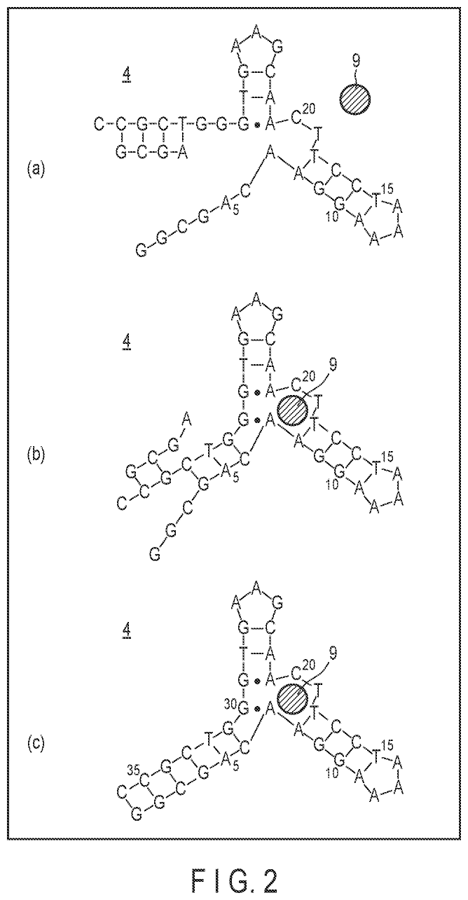

After the strand exchange reaction has completely proceeded, the free energy of the nucleic acid probe 4 is smaller than the free energy of the nucleic acid probe 4 having a metastable structure. Here, when a difference between the free energy of the nucleic acid probe 4 in a state where the strand exchange reaction proceeds and the free energy of the nucleic acid probe 4 after the strand exchange reaction has completely proceeded is defined as ΔE₂, the free energy of the nucleic acid probe 4 after the strand exchange reaction has completely proceeded is a level illustrated in part (c) of FIG. 3, and ΔE₁<ΔE₂. Therefore, the nucleic acid probe 4 in the strand exchange state proceeds more preferentially in a direction in which the first and second stems are formed than in a direction in which the first and fourth stems are formed. As a result, once the strand exchange occurs and the first and second stems are formed, the reaction becomes irreversible and it is difficult to return to the metastable structure.

Since the nucleic acid probe 4 is stably present in a state in which the first and second base sequences form a stem, the binding force for capturing the target substance is relatively large, but as described above, the capture of the target substance is a reversible reaction, and there is a case where the nucleic acid probe 4 is detached. However, since the nucleic acid probe 4 after completion of the strand exchange reaction is stable in a state where the first and second base sequences form a stem as described above, even when the captured target substance is detached from the binding site of the nucleic acid probe 4, the secondary structure of the nucleic acid probe 4 is hardly changed. On the other hand, the detached target substance can cause a further strand exchange reaction in the nucleic acid probe 4 by binding to the unreacted nucleic acid probe 4, and the number of the nucleic acid probes 4 in which the strand exchange reaction is caused is accumulated in the chemical sensor. Therefore, the chemical sensor according to the embodiment can detect a target substance at a concentration lower than the dissociation constant between the nucleic acid probe 4 and the target substance, and is preferable from the viewpoint of excellent detection sensitivity.

As a further embodiment, a donor fluorescent dye and an acceptor may be bound to any two of the first base sequence, the second base sequence, and the third base sequence, respectively. Here, the acceptor is a material having an absorption wavelength overlapping with an emission wavelength of the donor fluorescent dye. When the base sequence to which the donor fluorescent dye is bound and the base sequence to which the acceptor is bound are proximate to each other, fluorescence resonance energy transfer (FRET) occurs between the base sequences, such that emission of the donor dye in irradiation of the donor fluorescent dye with an excitation wavelength is attenuated. Here, when a fluorescent dye having an emission wavelength in a visible light range is used as the acceptor, emission of the acceptor dye is observed by irradiation of the donor fluorescent dye with an excitation wavelength.

For example, when the donor fluorescent dye is bound to the first base sequence and the acceptor is bound to the second base sequence, fluorescence of the donor fluorescent dye is observed before the nucleic acid probe 4 captures the target substance, but as the nucleic acid probe 4 captures the target substance and the strand exchange reaction proceeds, a state in which the binding of the first base sequence and the second base sequence is gradually formed and the emission intensity of the donor fluorescent dye is gradually decreased is observed. Therefore, when a donor fluorescent dye is bound to the first base sequence and an acceptor is bound to the second base sequence, a decrease in fluorescence intensity is detected, such that it is possible to detect whether a strand exchange reaction is caused, whether the target substance is captured, and whether the target substance is present in the sample. Similarly, the acceptor may be bound to the first base sequence and the donor fluorescent dye may be bound to the second base sequence. In addition, in a case where a fluorescent dye having an emission wavelength in the visible light range is used as the acceptor, the emission intensity of the acceptor dye may be measured.

In addition, a distance between the first base sequence and the third base sequence is increased after strand change because the third base sequence is released. In a case where a donor fluorescent dye is bound to such a first base sequence and an acceptor is bound to the third base sequence, it is possible to detect whether a strand exchange reaction is caused, whether the target substance is captured, and whether the target substance is present in the sample by detecting the change in fluorescence intensity.

### Second Embodiment

Hereinafter, a chemical sensor according to a second embodiment will be described. Note that description of similar members of as those of the chemical sensor described in the first embodiment will be omitted.

The chemical sensor according to the second embodiment is different from the chemical sensor according to the first embodiment in that an amino acid sequence of a polycation is bound to a second nucleic acid in addition to a fourth base sequence.

Note that the “polycation” in the present specification is, for example, a compound in which the number of cations per molecule in the liquid is equal to or greater than a numerical value of the base length of the double-stranded part formed by pairing the second base sequence of the first nucleic acid and the fourth base sequence of the second nucleic acid. For example, in a case where the double-stranded part formed by the second base sequence and the fourth base sequence is 4 bases, the polycation may have the number of cations of 4 or more per molecule.

An amino acid sequence of a polycation of the second nucleic acid is a sequence formed by binding lysine (Lys), arginine (Arg), and histidine (His), which are basic amino acid residues, to each other in an arbitrary combination in order.

In the second nucleic acid, the amino acid sequence of the polycation and the fourth base sequence may be directly bound to each other. For example, as illustrated in part (a) of FIG. 4, a second nucleic acid 20 contains a base sequence of GGCG in the 5′→3′ direction, and an amino acid sequence of a lysine hexamer may be bound to the 5′ end side to form a conjugate of Lys-Lys-Lys-Lys-Lys-Lys-GGCG. Here, when the second nucleic acid is DNA or RNA, the base sequence and the polycation can be bound to each other using a linker molecule, and when the second nucleic acid is a peptide nucleic acid (PNA), a copolymer can be produced using a peptide bond. In addition, a spacer sequence having a length of several bases may be contained between the polycationic sequence and the fourth base sequence.

In addition, in the second nucleic acid, the amino acid sequence of the polycation and the fourth base sequence may be indirectly bound to each other. For example, the amino acid sequence of the polycation may be bound to a PNA containing a fifth base sequence, a sixth base sequence complementary to the base sequence of the PNA may be bound to the fourth base sequence, and the polycation may be bound to the fourth base sequence by a double strand of the fifth and sixth base sequences.

A polycation 31 may be bound to a base sequence capable of forming a double strand with the second nucleic acid. For example, as illustrated in part (a) of FIG. 7, the polycation 31 contains an amino acid sequence KKKKKK, and may be a PNA formed by binding a base sequence TCTCTC. Alternatively, the polycation 31 may be a peptide consisting of an amino acid sequence KKKK (SEQ ID NO: 5: KKKK) or a peptide consisting of the amino acid sequence KKKKK (SEQ ID NO: 6: KKKKK) from the amino group toward the carboxy group.

For example, as illustrated in part (b) of FIG. 7, when a PNA of a sequence KKKKKKTCTCTC is used, a second nucleic acid 30 is a sequence consisting of GAGAGATGGCG (SEQ ID NO: 7) in order from the 5′ end. As illustrated in part (b) of FIG. 7, the second nucleic acid 30 of SEQ ID NO: 9 is a base sequence in which a base sequence 32 that complementarily binds to a base sequence TCTCTC (5′→3′ direction) of the polycation 31 and a fourth base sequence 23 composed of the sequence GGCG (5′→3′ direction) are bound to each other. Therefore, the second nucleic acid 30 and the polycation 31 are proximate to each other because they contain complementary base sequences.

In the first embodiment, it has been described that the target substance is captured at the binding site of the nucleic acid probe 4, such that the binding of the fourth base sequence of the second nucleic acid and the second base sequence of the first nucleic acid is gradually replaced by the binding of the first base sequence of the first nucleic acid and the second base sequence of the first nucleic acid by the strand exchange reaction. Here, referring to part (b) of FIG. 2, it is found that it is necessary to maintain a state in which three base sequences (that is, the first base sequence, the second base sequence, and the fourth base sequence) are proximate to each other in the strand exchange reaction.

In general, since the base sequence is negatively charged, an electric repulsive force is generated between a plurality of nucleic acids, and the electric repulsive force acts as an inhibitory action on bringing the nucleic acids proximate to each other. Therefore, it is considered that the strand exchange reaction described in part (b) of FIG. 2 is strongly subjected to the inhibitory action by the repulsive force between such negative charges.

Therefore, as illustrated in part (b) of FIG. 4, an amino acid sequence 24 of a polycation is further contained in the second nucleic acid 20, such that in a case where the strand exchange reaction is started by capturing the target substance, and the first base sequence 21, the second base sequence 22, and the fourth base sequence 23 are proximate to each other, the amino acid sequence 24 of the polycation can be proximate to these base sequences. Therefore, the repulsive force between the negative charges can be weakened, and the strand exchange reaction can be enhanced.

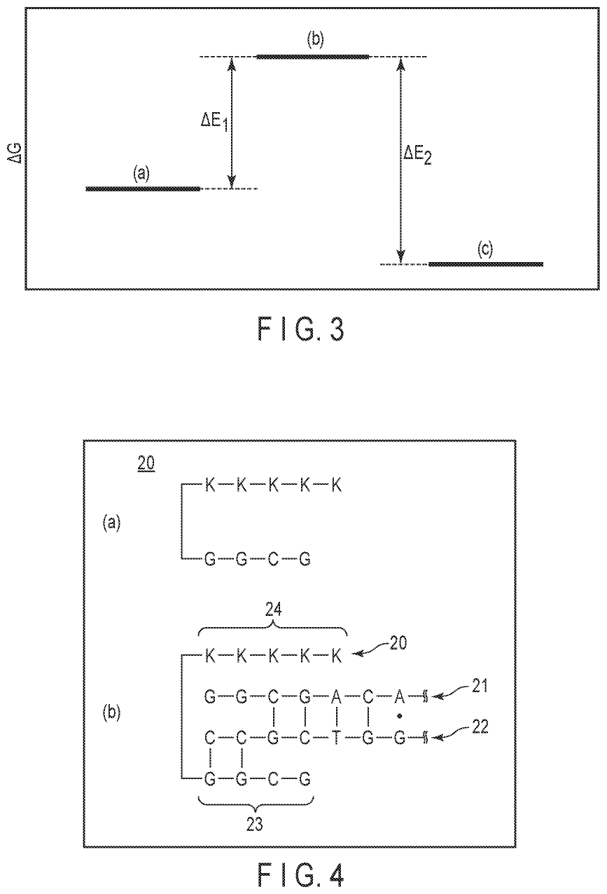

Referring to the free energy of the nucleic acid probe 4, the amino acid sequence 24 of the polycation of the second nucleic acid 20 can enhance the strand exchange reaction by reducing the energy barrier required for starting the strand exchange reaction from ΔE₁ to ΔE₁′ (see FIG. 5). However, it is necessary to pay attention to the point that an excessive polycation may cause a false positive signal because a reduction in energy barrier required for starting the strand exchange reaction also leads to an increase in the possibility that the strand exchange reaction starts caused by other influence different from the target substance. Therefore, when a material that enhances the strand exchange reaction (for example, an amino acid sequence of a polycation or the like) is added, it is preferable to adjust a desirable effect obtained by the addition to exceed an undesirable side reaction. For example, it can be adjusted by optimizing an addition amount of a material that enhances the strand exchange reaction, by configuring a molecular structure of the material that enhances the strand exchange reaction to contain a necessary minimum number of cations, and the like.

Furthermore, in the chemical sensor of the second embodiment, when the strand exchange reaction is completed, the second nucleic acid is dissociated from the first nucleic acid, such that the polycation bound to the second nucleic acid is released from the nucleic acid probe 4. The release of the polycation can prevent a strand exchange reaction of a reverse reaction, and separation of positive charges associated with the release of the polycation can be obtained as a strong detection signal.

### Third Embodiment

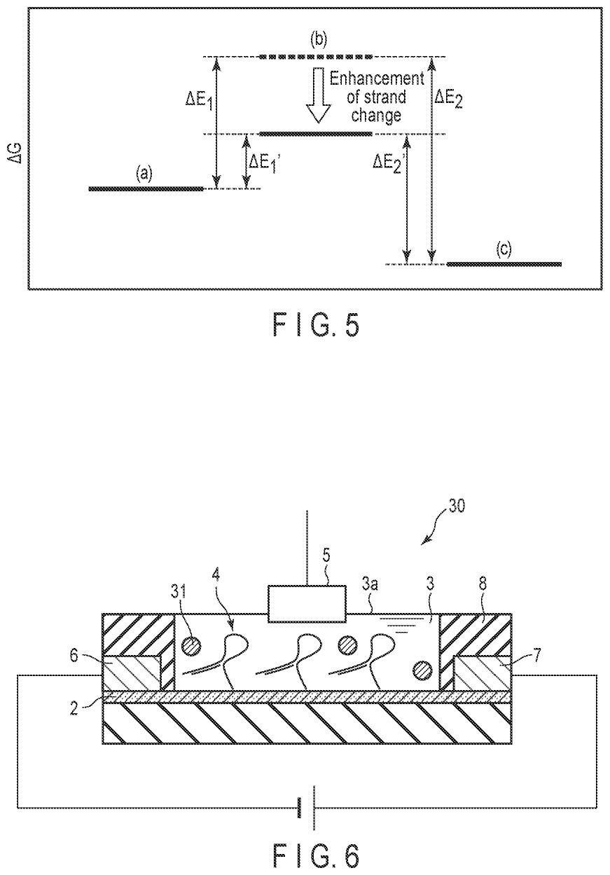

A chemical sensor 30 according to a third embodiment will be described in detail with reference to FIG. 6. Note that, in FIG. 6, members similar to those in FIG. 1 described in the first embodiment are denoted by the same reference numerals, and description thereof is omitted.

The chemical sensor 30 according to the third embodiment is different from the chemical sensor of the first embodiment in that a released polycation 31 is further included in a liquid film 3.

In the chemical sensor of the second embodiment, the amino acid sequence 24 of the polycation is contained in the second nucleic acid 20, but in the chemical sensor 30 of the third embodiment, a repulsive force between negative charges as counter ions is weakened by containing the released polycation 31 as a solute in a liquid film.

Therefore, similar to the second embodiment, the chemical sensor 30 of the third embodiment can easily start a strand exchange reaction by bringing the released polycation 31 proximate to a first base sequence 21, a second base sequence 22, and a fourth base sequence 23 of a second nucleic acid 30.

### Fourth Embodiment

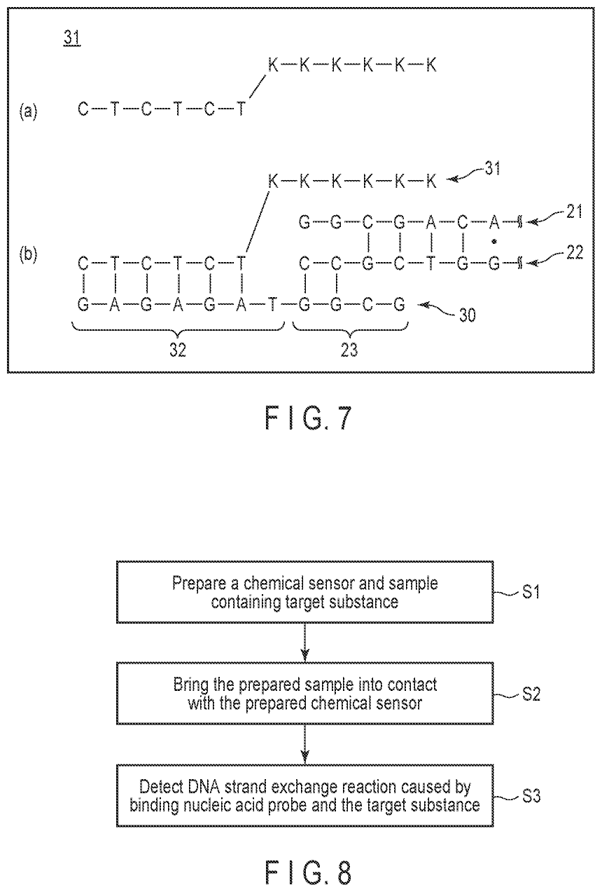

As a fourth embodiment, a method for detecting a target substance using the chemical sensor described in each of the first to third embodiments is provided. As illustrated in FIG. 8, the detection method includes: (S1) preparing the chemical sensor described in each of the first to third embodiments and a sample having a possibility of containing a target substance; (S2) bringing the prepared sample into contact with the chemical sensor prepared in (S1); and (S3) detecting a DNA strand exchange reaction that is caused by binding of a nucleic acid probe and the target substance.

The target substance may be a material in any type, for example, a gaseous, liquid, or particulate material. In addition, a sample containing such a target substance may be a gas or a liquid. In a case where the sample is a gas, in Step (S2) described above, the sample may be brought into contact with a surface of the liquid film included in the chemical sensor so as to be sprayed, or the liquid film may be brought into contact so as to be bubbled. In a case where the sample is a liquid, the sample can be brought into contact with the liquid film by adding the sample to the liquid film.

Step (S3) described above can be performed by detecting a change in secondary structure of a nucleic acid probe 4 due to a strand exchange reaction with a sensor element of the chemical sensor. Here, when the detection is performed, a temperature of a liquid film 3 included in a chemical sensor 1 is adjusted to a temperature lower than a melting temperature Tm of a first nucleic acid. By adjusting the temperature to such a temperature, in the first nucleic acid after strand exchange, a state in which a first base sequence and a second base sequence are bound to each other becomes stable.

When a structural change or a change in electrical characteristics caused by release occurs in the nucleic acid probe 4, the change is detected as a signal change of a sensor element 2. Here, in a case where the sensor element is an FET element, Step (S3) described above can be performed by detecting a change in electrical characteristics of the FET. As the change in electrical characteristics of the FET, there is a method for detecting a change in drain current and a change in IdVg characteristic.

For example, in a case where the sensor element includes a carbon allotrope film, as electrical characteristics of the carbon allotrope film, in particular, graphene exhibits a unique band structure in which a conduction band and a valence band intersect with each other at one point without having a band gap (this point is referred to as a “Dirac point”). In a case where the Fermi level of a carbon allotrope film 2 is at a Dirac point, a carrier density is the lowest, and electrical resistance of graphene is the highest. In a case where the Fermi level is lower than the Dirac point, the Fermi level is located in the valence band, and P-type conduction using holes as carriers is exhibited. In a case where the Fermi level is further decreased, the density of holes is increased, such that the conductivity of graphene is improved. On the other, in a case where the Fermi level of the carbon allotrope film is higher than the Dirac point, the Fermi level is located in the conduction band, such that N-type conduction using electrons as carriers is exhibited. When the Fermi level is further increased, the density of holes is increased, such that the conductivity of graphene is improved.

In the chemical sensor 1 including the carbon allotrope film having the properties described above as the sensor element 2, a magnitude of the drain current obtained by scanning a gate voltage is a minimum value at a gate voltage at which the Fermi level is equal to the Dirac point, and the magnitude tends to be increased as the Fermi level moves away from the Dirac point by changing the gate voltage. Such an IdVg characteristic of the carbon allotrope film can be expressed by a V-shaped curve. In addition, a point at which the magnitude of the drain current has a minimum value on the V-shaped curve is referred to as a “charge neutral point (CNP)”.

Here, when a surface of the carbon allotrope film is positively charged, a negative charge is induced in the carbon allotrope film, such that the Fermi level is increased. Since this phenomenon is applied as a bias with respect to the gate voltage, the V-shaped curve of the IdVg characteristic of the carbon allotrope film was shifted in a low gate voltage direction. Conversely, in a case where the surface of the carbon allotrope film is negatively charged, the V-shaped curve of the IdVg characteristic shifts in a high gate voltage direction. In addition, the shift of the V-shaped curve can also be regarded as a shift of the CNP. Therefore, it is possible to detect a change in surface charge of the carbon allotrope film by observing the V-shaped curve of the IdVg characteristic and the shift of the CNP in the gate voltage direction. When the secondary structure of the nucleic acid probe 4 is changed, nucleic acids having a negative charge are proximate to or separated from each other, such that this can be read as a change in surface charge.

As another measurement method for detecting a change in electrical characteristics of the carbon allotrope film, for example, there is also a method of fixing a gate voltage and measuring a temporal change in drain current. In a case where the gate voltage applied to the chemical sensor 1 is set to a constant value, a constant drain current value is output when the surface charge of the carbon allotrope film is not changed. In a case where the secondary structure of the nucleic acid probe 4 is changed from such a constant state, a change in drain current occurs due to a change in surface charge due to the proximity and separation of the negative charges of the nucleic acid in the same manner as described above.

Alternatively, a molecular structure change of the nucleic acid probe 4 may be detected by providing the donor fluorescent dye and the acceptor as described in the first embodiment to the nucleic acid probe 4 and detecting a change in fluorescence intensity by, for example, the sensor element 2, a fluorescence detection device (not illustrated), or the like.

In the method according to the fourth embodiment, the presence of a target substance can be detected with high sensitivity because the target substance is detected by inducing a strand exchange reaction as described in the first to third embodiments.

Note that in Steps (S1) to (S3) may be continuously executed, or may include any step related to, for example, use of the sensor between the steps.

### Fifth Embodiment

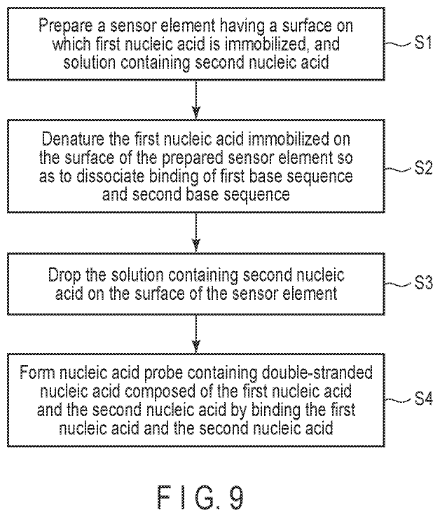

As a fifth embodiment, a method for manufacturing the chemical sensor described in each of the first to third embodiments is provided. As illustrated in FIG. 9, the manufacturing method includes: (S1) preparing a sensor element that has a surface on which a first nucleic acid is immobilized and is coated with a liquid film, the first nucleic acid having a stem portion formed by binding a first base sequence and a second base sequence, and a solution containing a second nucleic acid; (S2) denaturing the first nucleic acid immobilized on the surface of the sensor element prepared in (S1) so as to dissociate the binding of the first base sequence and the second base sequence; (S3) dropping a solution containing an excessive amount of the second nucleic acid compared to the first nucleic acid on the surface of the sensor element including the first nucleic acid; and (S4) forming a nucleic acid probe containing a double-stranded nucleic acid composed of a first nucleic acid and a second nucleic acid by binding the first nucleic acid and the second nucleic acid.

In (S1) of the method according to the embodiment, a sensor element that has a surface on which a first nucleic acid is immobilized is prepared. Since the first nucleic acid is a nucleic acid that autonomously forms a stable higher-order structure, the first nucleic acid immobilized on the surface of the prepared sensor element also has a stable higher-order structure.

Note that, as described in the first embodiment, the immobilization of the first nucleic acid on the surface of the sensor element can be performed by making a material constituting a surface 2a of a sensor element 2 a material capable of binding to a first nucleic acid or a second nucleic acid of a nucleic acid probe 4. For example, when the sensor element 2 is formed of graphene or gold, the nucleic acid probe 4 is immobilized because of non-specific adsorption with the nucleic acid. More preferably, a functional group that binds to or absorbs a material constituting the surface 2a of the sensor element 2 is modified on the nucleic acid probe 4, and the nucleic acid probe 4 may be immobilized by binding to or adsorbing the sensor element surface 2a and the functional group. For example, in a case where the sensor element 2 is graphene, when a polycyclic aromatic system, for example, pyrene is modified as the functional group, the nucleic acid probe 4 is strongly immobilized on the graphene sensor element surface 2a by a π-π interaction between the polycyclic aromatic system and the graphene. Alternatively, in a case where the sensor element 2 is gold, when a thiol group is modified as the functional group, the thiol group strongly binds to gold, such that the nucleic acid probe 4 is strongly immobilized on the gold surface 2a of the sensor element 2. Note that the functional group modification of the nucleic acid may be performed by nucleic acid synthesis by a phosphoramidite method using an amidite reagent having a desired functional group.

In (S2) of the method according to the embodiment, the first nucleic acid that autonomously forms a stable higher-order structure is denatured on the sensor element. The denaturation may be performed in a method known in the art that is capable of dissociating complementary strands of nucleic acids. Examples of the known method include denaturation by heat and denaturation by a reduction in salt concentration.

In order to executing Step (S2), the chemical sensor may include a heating device for thermal denaturation. The heating device may be configured to heat a liquid film to a melting temperature Tm of the first nucleic acid, and may employ a known heat generation mechanism such as a resistance heating element.

In addition, the chemical sensor may include a device configured to supply a low salt concentration solution to the surface of the sensor element as a device for executing Step (S2). The device configured to supply the low salt concentration solution includes, for example, a container for storing the low salt concentration solution, and a flow path and a pump configured to supply the low salt concentration solution to the surface of the sensor element.

In (S3) of the method according to the present embodiment, a solution containing a second nucleic acid is dropped on the surface of the sensor element on which the first nucleic acid denatured in Step (S2) is immobilized. In a case where dissociation of the first and second base sequences in Step (S2) is performed by heating, it is preferable to continue to perform heating until Step (S3) in order to suppress recombination of the first and second base sequences. In addition, in a case where Step (S2) is executed by supplying a low salt concentration solution, when an excessive amount of the second nucleic acid is contained in the low salt concentration solution supplied in Step (S2) and a high salt concentration solution containing an excessive amount of the second nucleic acid is dropped in Step (S3), an excessive amount of the second nucleic acid is always present in a process of changing the salt concentration, and therefore recombination of the first and second base sequences can be suppressed.

In addition, Step (S3) may be performed simultaneously with the start of Step (S2) regardless of the denaturation method. In other words, the second nucleic acid may be further contained in the liquid film included in the sensor element prepared in Step (S1), and the second nucleic acid may be subjected to Step (S2) together with the first nucleic acid.

In (S4) of the method according to the embodiment, a nucleic acid probe containing a double-stranded nucleic acid composed of a first nucleic acid and a second nucleic acid is formed by binding a first nucleic acid and a second nucleic acid. The binding of the first nucleic acid and the second nucleic acid can be performed by a technique known in the art capable of binding complementary strands of nucleic acids. Examples of the known method include binding by cooling to a melting temperature or lower and binding by increasing the salt concentration.

In a case where a heating device is used in Step (S3), the chemical sensor includes a cooling device in order to execute Step (S4). The cooling device is configured such that the temperature of the liquid film is lower than the melting temperature Tm of the first nucleic acid, and for example, a known heat absorbing mechanism such as a Peltier element or a known heat discharging mechanism such as a heat sink or a heat pump may be employed.

In addition, in a case where a device or a flow path configured to supply a low salt concentration solution is used in Step (S3), a device configured to supply a high salt concentration solution to the sensor element surface 2a is further provided as a device for executing Step (S4). The same device may include, for example, a container for storing a high salt concentration solution, and a flow path and a pump configured to supply the high salt concentration solution to the sensor element surface.

In addition to the method according to the embodiment, the nucleic acid probe 4 may be formed by containing a double-stranded nucleic acid obtained by binding a first nucleic acid and a second nucleic acid in a liquid film and immobilizing the double-stranded nucleic acid on the surface 2a of the sensor element 2. According to such a method, it is required to prepare a double-stranded nucleic acid obtained by binding the first nucleic acid and the second nucleic acid, but the operation on the chemical sensor may be immobilizing the double-stranded nucleic acid on the chemical sensor.

Hereinafter, the nucleic acid probe described in the embodiment will be described using experimental data.

### EXAMPLE

### Example 1

Measurement of Temporal Change in Drain Current by Increasing Concentration of Target Substance Stepwise

Preparation of Chemical Sensor

A sensor 30 having the same structure as in FIG. 6 was prepared. A sensor element included in the sensor 30 is a single-layer graphene film. A voltage application circuit is connected to the sensor 30, and a gate voltage can be applied between a gate electrode and a source electrode, and a drain voltage can be applied between a drain electrode and the source electrode. The drain current flowing through the sensor 30 by the drain voltage exhibits an FET characteristic which changes into a V shape by a change in gate voltage. In addition, the sensor 30 includes an ammeter (not illustrated) for measuring the drain current.

As a nucleic acid probe 4, the double-stranded nucleic acid illustrated in part (a) of FIG. 2, that is, a double-stranded nucleic acid in which a first nucleic acid as MN4 and a second nucleic acid as a short-chain complementary strand composed of a base sequence GCGA were bound to each other was used.

In Example 1, a target substance of the nucleic acid probe 4 is methyl benzoate (hereinafter, referred to as MB) that is a material derived from cocaine. As samples containing the same target substance, samples having four different concentrations (specifically, a liquid sample containing 0 μM of MB, a liquid sample containing 1 μM of MB, a liquid sample containing 10 μM of MB, and a liquid sample containing 100 μM of MB) were prepared and used.

In addition, in Example 1, a polycation composed of an amino acid sequence KKKKK (SEQ ID NO: 10) was contained in a liquid film 3 included in the sensor 30.

Change in Drain Current Under Constant Gate Voltage

A constant gate voltage was applied to the sensor 30 having the configuration described above, and a temporal change in drain current when a concentration of the target substance was increased stepwise was measured. Note that, as shown in Table 1, a liquid sample having an MB concentration of 0 μM was supplied from a start of measurement to T1, a liquid sample having an MB concentration of 1 μM was supplied for T1 to T2, a liquid sample having an MB concentration of 10 μM was supplied for T2 to T3, a liquid sample having an MB concentration of 100 μM was supplied for T3 to T4, and a liquid sample having an MB concentration of 0 μM was supplied after T4. Each sample was supplied by replacement of the liquid film 3 of the chemical sensor with a pipette.

TABLE 1

| Measurement time | Concentration of |
|---|---|
| (second) | methyl benzoate (MB) |
| 0 to T1 | 0 |
| T1 to T2 | 1 |
| T2 to T3 | 10 |
| T3 to T4 | 100 |
| T4 or more | 0 |

As described below, the measurement of the temporal change in drain current was temporarily interrupted when t1, t2, t3, t4, and t5 have elapsed from the start of the measurement, and the IdVg characteristic of the graphene film at each time point was measured by scanning the gate voltage. After the measurement of the IdVg characteristic at each time point, the gate voltage was quickly returned, and the measurement of the temporal change in drain current was resumed. For example, the measurement of the temporal change in drain current with the gate voltage set to 0 mV was temporarily interrupted at time T1, the gate voltage was scanned between −500 mV to +500 mV to measure the IdVg characteristic, and then the gate voltage was set to 0 mV again to resume the measurement of the temporal change in drain current. At that time, the time point at which the measurement was resumed was set to time T1. Note that a gate voltage of 0 mV does not mean that no gate voltage is applied, but means that the source electrode and the drain electrode are fixed at the same potential. Since there is a potential difference between the gate electrode and the liquid film at which the electrochemical reaction is in an equilibrium state, a potential difference between the liquid film and the graphene is not 0 mV.

Measurement of IdVg Characteristic of Graphene Film

As described above, when t1, t2, t3, t4 and t5 have passed after the start of measurement, the measurement of the temporal change in drain current was temporarily interrupted, and the IdVg characteristic of the graphene film at each time point was measured by scanning the gate voltage. In the measurement of the IdVg characteristic, the scanning of the gate voltage was performed in a range of −500 mV to +430 mV, but since important information was not included at 0 mV or less, a range of 0 mV to 430 mV was enlarged and illustrated.

Results

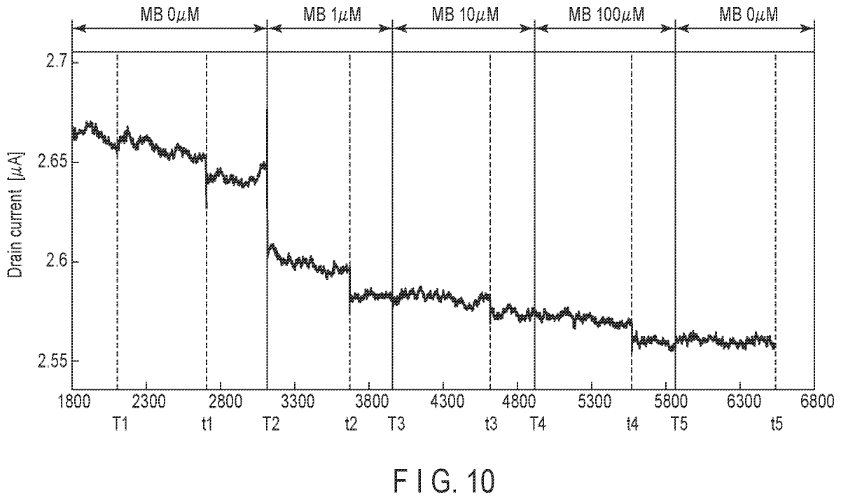

The measurement results of the temporal change in drain current are illustrated in FIG. 10. There is no correlation with the timing of liquid replacement, and the baseline of the drain current is gradually decreased over time, which is a drift specific to the measurement of electrochemical phenomenon. On the other hand, at time T1 when the methyl benzoate concentration was changed from 0 μM to 1 μM, a rapid decrease in drain current, which was clearly different from the baseline movement, was observed. Such a rapid change was not observed in the subsequent liquid replacement, and the drain current was not recovered (increased) even when the methyl benzoate concentration was finally returned to 0 μM.

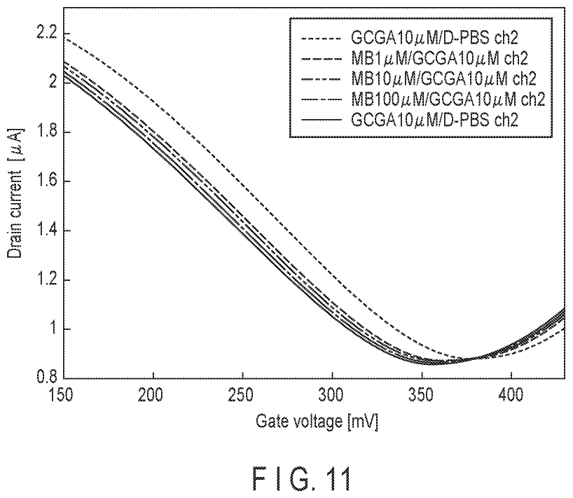

The measurement results of the IdVg characteristic are illustrated in FIG. 11. Referring to FIG. 11, it can be seen that the V-shaped curve of the IdVg characteristic was shifted in the low gate voltage direction particularly in the measurement at t2. That is, it was suggested that at time t2, a negative charge was proximate to the graphene film as the sensor element. In addition, almost no difference was observed in the V-shaped curve of the IdVg characteristic measured at t3, t4, and t5 time points.

With reference to the above results, a significant decrease in drain current at time T1 is evidently due to the addition of 1 μM of MB solution. In addition, the dissociation constant between MN4 and MB as target substances were about 100 μM, which showed that the chemical sensor according to the present embodiment could detect even a target substance at a concentration lower than the dissociation constant by one digit or more. This is considered to be because, as described above, the target substance captured by the nucleic acid probe is detached from the binding site and rebinds to another unreacted nucleic acid probe.

Furthermore, since the value of the drain current was not recovered even when a sample having MB of 0 μM was added after time T4, it was suggested that the higher-order structure after the strand exchange reaction was stable, and the reaction of changing from the metastable state to the stable structure through the strand exchange was irreversible.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A chemical sensor comprising:
a nucleic acid probe for capturing a target substance;
a sensor element that has a surface on which the nucleic acid probe is immobilized; and
a liquid film that covers the sensor element,
wherein the nucleic acid probe is a double-stranded nucleic acid composed of a first nucleic acid and a second nucleic acid bound to the first nucleic acid,
the first nucleic acid is a single-stranded nucleic acid containing a first base sequence, a second base sequence consisting of a base sequence complementary to the first base sequence, and a third base sequence having one end bound to the first base sequence and the other end bound to the second base sequence,
the second nucleic acid is a single-stranded nucleic acid containing a fourth base sequence consisting of a base sequence complementary to a part of the second base sequence of the first nucleic acid, and
a base sequence constituting a binding site that captures the target substance is included in the third base sequence.

2. The chemical sensor according to claim 1, wherein when the third base sequence captures the target substance, the first nucleic acid and the second nucleic acid are dissociated, and a secondary structure having a stem portion formed by pairing the first base sequence and the second base sequence and a loop portion or a bulge structure containing the third base sequence is formed in the first nucleic acid.

3. The chemical sensor according to claim 1, wherein a base length of the fourth base sequence is shorter than a base length of the second base sequence.

4. The chemical sensor according to claim 1, wherein the first nucleic acid of the nucleic acid probe contains a spacer sequence of one base, two bases, or three bases between the base sequence constituting the binding site of the third base sequence and the second base sequence.

5. The chemical sensor according to claim 1, wherein a spacer of one base is contained between the base sequence constituting the binding site of the third base sequence and the first base sequence.

6. The chemical sensor according to claim 1, wherein the first nucleic acid dissociated from the second nucleic acid forms a higher-order structure by capturing the target substance with the third base sequence.

7. The chemical sensor according to claim 1, wherein the secondary structure of the first nucleic acid formed by capturing the target substance with the third base sequence is a hairpin loop, an internal loop, a bulge, a pseudoknot, a 3 way junction, or a 4 way junction.

8. The chemical sensor according to claim 1, wherein the sensor element is any one of a graphene FET, an ISFET, an SPR, and a QCM.

9. The chemical sensor according to claim 1, wherein the nucleic acid probe is immobilized on the surface of the sensor element by immobilizing one of the first nucleic acid and the second nucleic acid on the surface of the sensor element.

10. The chemical sensor according to claim 1, wherein the fourth base sequence includes a base sequence mismatched with the second base sequence.

11. The chemical sensor according to claim 1, wherein the fourth base sequence of the second nucleic acid further includes a base sequence that does not pair with the second base sequence, or the second base sequence includes a base sequence that does not pair with the fourth base sequence, and
the chemical sensor includes the nucleic acid probe containing a double strand having a bulge structure in which the second base sequence of the first nucleic acid and the fourth base sequence of the second nucleic acid are bound to each other.

12. The chemical sensor according to claim 1, wherein the second nucleic acid is a PNA in which the fourth base sequence and an amino acid sequence of a polycation are bound to each other.

13. The chemical sensor according to claim 1, wherein the liquid film contains a PNA containing an amino acid of a polycation or an amino acid sequence of a polycation.

14. The chemical sensor according to claim 13, wherein the amino acid sequence of the polycation is a part of a PNA containing a base sequence complementary to a base sequence of a part of the second nucleic acid, and
the polycation binds to a base sequence of a part of the second nucleic acid to form a double strand with the second nucleic acid.

15. The chemical sensor according to claim 1,
wherein a donor fluorescent dye and an acceptor are bound to any two of the first base sequence, the second base sequence, and the third base sequence, respectively, and
fluorescence resonance energy transfer (FRET) occurs between the base sequence to which the donor fluorescent dye is bound and the base sequence to which the acceptor is bound.

16. A method for detecting a target substance using the chemical sensor according to claim 1, the method comprising:
(S1) preparing the chemical sensor according to claim 1 and a sample containing a target substance;
(S2) bringing the sample into contact with the liquid film of the chemical sensor; and
(S3) detecting a DNA strand exchange reaction that is caused when the target substance is captured by the nucleic acid probe.

17. A method for manufacturing the chemical sensor according to claim 1, the method comprising:
(S1) preparing a sensor element that has a surface on which the first nucleic acid is immobilized and is coated with the liquid film, the first nucleic acid having a stem portion formed by binding the first base sequence and the second base sequence, and a solution containing the second nucleic acid;
(S2) denaturing the first nucleic acid immobilized on the surface of the sensor element prepared in (S1) so as to dissociate the binding of the first base sequence and the second base sequence;
(S3) dropping the solution containing the second nucleic acid on the surface of the sensor element; and
(S4) forming the nucleic acid probe by binding the first nucleic acid and the second nucleic acid.

18. The method according to claim 17,
wherein the sensor element prepared in (S1) further includes a heating device configured to heat the solution constituting the liquid film and a cooling device configured to cool the solution constituting the liquid film,
(S2) is performed by heating the solution constituting the liquid film by the heating device to denature the first nucleic acid, and
(S4) is performed by cooling the solution constituting the liquid film by the cooling device.

19. The method according to claim 17, wherein the chemical sensor further includes a device configured to supply a low salt concentration solution to the surface of the sensor element and a device configured to supply a high salt concentration solution to the surface of the sensor element, and
the method comprises:
after (S1) and before (S2), replacing the solution constituting liquid film with the low salt concentration solution supplied from the device configured to supply the low salt concentration solution to the surface of the sensor element; and
after (S4), replacing the solution constituting the liquid film with the high salt concentration solution supplied from the device configured to supply the high salt concentration solution to the surface of the sensor element.
